(19) United States
(12) Patent Application Publication
(10) Pub. No.: US 2003/0212422 A1
(43) Pub. Date: Nov. 13, 2003

(54) Ultrasonic soft tissue cutting and coagulation systems with movable vibrating probe and fixed receiving clamp

(76) Inventors: Paul Fenton, Marblehead, MA (US); Francis Harrington, Peabody, MA (US); Paul Westhaver, Newburyport, MA (US)

Correspondence Address: Mark G. Lappin, McDermott, Will & Emery, 28 State Street, Boston, MA, 02109, US

(21) Appl. No.: 10/436,857

(22) Filed: May 13, 2003

Related U.S. Application Data
(60) Provisional application No. 60/380,177, filed on May 13, 2002.

(51) Int. Cl. A61B 17/32

(52) U.S. Cl. 606/169; 606/37

(57) ABSTRACT

An ultrasonic soft tissue cutting and coagulation system has a movable ultrasonic probe member connected to a stationary clamp jaw. The probe member is movable between an open position, spaced from a tissue engaging surface of the clamp jaw, to a clamped position in which the probe member is moved toward the tissue engaging surface so as to capture tissue therebetween.

### CROSS-REFERENCE TO RELATED APPLICATIONS

The present application claims priority to provisional U.S. patent application serial No. 60/380,177, filed on May 13, 2002, which is assigned to the assignee of the present application and incorporated herein by reference.

### STATEMENT REGARDING FEDERALLY SPONSORED RESEARCH

Not Applicable

### REFERENCE TO MICROFICHE APPENDIX

Not Applicable

### BACKGROUND OF THE INVENTION

For many years, ultrasonic surgical instruments have been used for soft tissue cutting and coagulation. These ultrasonic instruments include ultrasonic transducers which convert the electric energy supplied by a generator into ultrasonic frequency vibratory energy, which can then be applied to the tissue of a patient. Ultrasonic surgical instruments use relatively high-power, low-frequency vibratory energy, typically at a frequency range of about 20 kHz to about 100 kHz.

In general, ultrasonic tissue cutting and coagulation systems include a probe or horn that is coupled to the ultrasonic transducers, and thus can be made to vibrate at ultrasonic frequencies. The ultrasonically vibrating probe is then applied to the tissue, in order to transmit ultrasonic energy to the tissue. In this way, the contacted tissue can be cut or coagulated.

The mechanism through which the ultrasonic probe and the tissue interact, i.e. the physics of ultrasonic soft tissue cutting and coagulation, is not completely understood, however various explanations have been provided by researchers over the years. These explanations include descriptions of mechanical effects and thermal effects. The mechanical viewpoint states that when the vibrating tip of the ultrasonic probe generates short-range forces and pressures, which are sufficient to dislodge cells in the tissue, and break up the tissue structures. Various types of forces are postulated as contributing to the rupture of the tissue layer, for example the impact forces resulting from the direct contact of the vibrating tip with tissue, and the shear forces that are the result of the differences in force levels across tissue boundaries. Some energy may be lost due to frictional heating, and by the heating caused by the absorption of acoustic energy by tissue.

Thermal effects may include frictional heat, generated by the ultrasonically vibrating tip, in an amount sufficient to melt a portion of the contacted tissue. Alternatively, the tissue may absorb the vibratory energy, which it then converts into heat. The generated heat may be used to coagulate a blood vessel, by way of example. Other effects that have been postulated in order to explain the probe-tissue interaction include cavitational effects. The cavitation viewpoint postulates that the coupling of ultrasonic energy onto tissue results in the occurrence of cavitation in tissue, namely the formation of gas or vapor-filled cavities or bubbles within the tissue, which may oscillate and propagate. A combination of mechanical, thermal, and cavitational effects may result in the desired surgical outcomes, such as cutting and coagulation.

A number of ultrasonic soft tissue cutting and coagulating systems have been disclosed in the prior art. For example, U.S. Pat. No. 5,322,055 (the “'055 patent”), entitled “Clamp Coagulator/Cutting System For Ultrasonic Surgical Instruments,” discloses ultrasonic surgical instruments having a non-vibrating clamp for pressing tissue against an ultrasonically vibrating blade, for cutting, coagulating, and blunt-dissecting of tissue. The '055 patent issued to T. W. Davison et al. on Jun. 21, 1994, and is assigned on its face to Ultracision, Inc.

The '055 patent relates to ultrasonic surgical instruments having a non-vibrating clamp for pressing tissue against an ultrasonically vibrating blade, for cutting, coagulating, and blunt-dissecting of tissue. A handpiece enclosing an ultrasonic transducer is connected to the blade. When ultrasonically activated, the blade undergoes longitudinal mode vibrations, parallel to the blade edge. A clamp accessory, including a clamp jaw, is releasably connected to handpiece. The blade is used in conjunction with the clamp jaw, to apply a compressive force to the tissue in a direction normal to the direction of vibration. In a preferred embodiment of the invention, a clamp jaw actuation mechanism, for example a scissors-like grip, actuates a pivoted clamp jaw to compress and bias tissue against the ultrasonic energy-carrying blade, in a direction normal to the longitudinal vibratory movement of the blade.

U.S. Pat. No. 6,036,667 (the “'667 patent”), entitled “Ultrasonic Dissection and Coagulation System,” issued to R. Manna et al. on Mar. 14, 2000, and is assigned on its face to United States Surgical Corporation and to Misonix Incorporated.

The '667 patent discloses an ultrasonic dissection and coagulation system for surgical use. The ultrasonic system includes a housing, and an elongated body portion extending from the housing. The housing encloses an ultrasonic transducer, which is operatively connected to a cutting blade by a vibration coupler. The cutting blade has a cutting surface which is angled with respect to the longitudinal axis (“LA”) of the elongated body portion, i.e. with respect to the axis of ultrasonic vibration. A clamp member, having a tissue contact surface, is positioned adjacent to the blade. The clamp member is movable from an open position in which the tissue contact surface of the clamp is spaced from the cutting surface of the blade, to a clamped position in which the tissue contact surface of the clamp is in close juxtaposed alignment with the cutting surface to clamp tissue therebetween.

U.S. Pat. No. 6,056,735 (the “'735 patent”), entitled “Ultrasound Treatment System,” relates to ultrasonic treatment systems, including endoscopic systems and aspiration systems, for treating living tissue. The '735 patent issued to M. Okada et al. on May 2, 2000, and is assigned on its face to Olympus Optical Co., Ltd.

The '735 patent features an ultrasonic treatment system having a handpiece that encloses ultrasonic transducers, and a probe connected to the transducers and serving as an ultrasonic energy conveying member. A treatment unit of the ultrasonic treatment system includes a stationary, distal member, to which ultrasonic vibrations are conveyed by the probe, and a movable, holding member. The holding member clamps living tissue, in cooperation with the fixed distal member. A scissors-like manipulating means manipulates the treatment unit to clamp or free living tissue. In a preferred embodiment, a turning mechanism is provided for turning the treatment unit relative to the manipulating means, with the axial direction of the transducers as a center.

All of the prior art ultrasonic systems described above require that the ultrasonically vibrating probe, i.e. the component of the system that receives ultrasonic energy and transmits the ultrasonic energy to the tissue, be stationary with respect to the clamp or other holding member.

In order to increase the versatility and maneuvrability of the surgical systems, however, it is desirable that an ultrasonic system be provided that includes an ultrasonic probe that is movable with respect to a fixed receiving clamp. A movable ultrasonic probe member would also be able to achieve a greater variety of surgical effects.

### SUMMARY OF THE INVENTION

The present invention is directed to an ultrasonic soft tissue cutting and coagulation system, which has a movable ultrasonic probe member connected to a stationary clamp jaw.

The present invention features an ultrasonic surgical instrument having an ultrasonic transducer for generating ultrasonic vibrations. A probe member is connected to said ultrasonic transducer for receiving ultrasonic vibrations therefrom. A stationary clamp jaw is provided. The clamp jaw includes a tissue engaging surface. The probe member is movably connected to the clamp jaw. The probe member is movable between an open position spaced from the tissue engaging surface of the clamp jaw, to a clamped position in which the probe member is moved toward the tissue engaging surface so as to capture tissue therebetween. When the surgical instrument is used for cutting tissue, the probe member may include a cutting surface.

### BRIEF DESCRIPTION OF THE DRAWINGS

The invention can be more fully understood by referring to the following detailed description taken in conjunction with the accompanying drawings, in which:

FIG. 1 illustrates an overall schematic view of an ultrasonic surgical system, constructed in accordance with the present invention.

FIG. 2 illustrates a probe-jaw assembly for an ultrasonic surgical system constructed in accordance with one embodiment of the present invention, in which the probe member has an operative surface that is substantially convex-shaped with respect to a longitudinal axis (“LA”) thereof, and the clamp jaw has an operative surface that is substantially concave-shaped with respect to a longitudinal axis (“LA”) thereof.

FIG. 3 illustrates another embodiment of a probe-jaw assembly for an ultrasonic surgical system constructed in accordance with one embodiment of the present invention, in which the probe member has an operative surface that is substantially concave-shaped with respect to a longitudinal axis (“LA”) thereof, and the clamp jaw has an operative surface that is substantially convex-shaped with respect to a longitudinal axis (“LA”) thereof.

FIG. 4 illustrates a probe-jaw assembly for an ultrasonic surgical system constructed in accordance with another embodiment of the present invention, in which the ultrasonic probe member is movable in a direction parallel to the longitudinal vibrations.

FIG. 5 illustrates a probe-jaw assembly for an ultrasonic surgical system constructed in accordance with another embodiment of the present invention, in which the ultrasonic probe member can be moved rotatably with respect to the fixed jaw.

FIG. 6 illustrates a probe-jaw assembly for an ultrasonic surgical system constructed in accordance with another embodiment of the present invention, in which the ultrasonic probe member can be moved so as to pass through a matching orifice in the fixed jaw.

### DETAILED DESCRIPTION

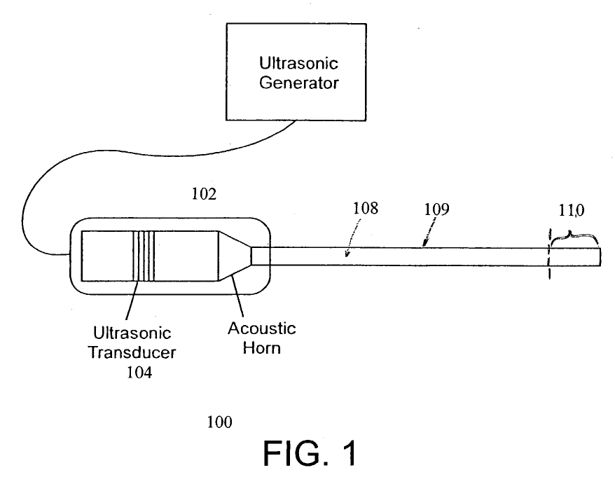

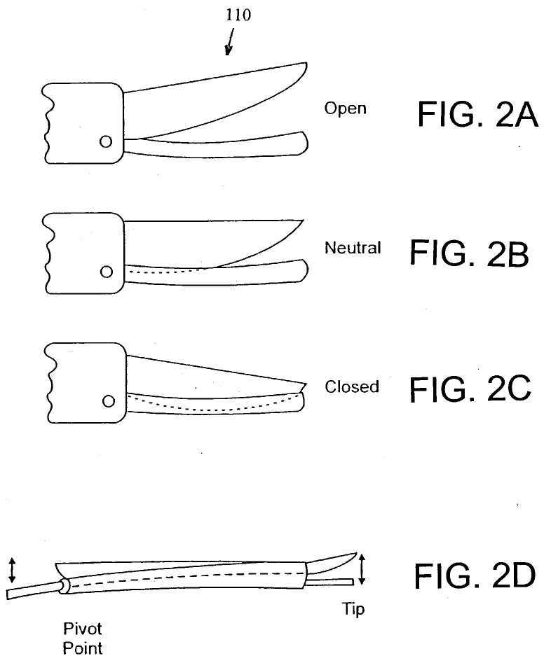

FIG. 1 illustrates an overall schematic view of an ultrasonic soft tissue cutting and coagulating system 100, constructed in accordance with the present invention. The system include a handpiece 102, an ultrasonic energy transmission guide (or horn) 108 covered by a sheath 109, and a tip assembly 110 connecting to a ultrasonic probe-jaw assembly (shown in FIGS 2-6), extending from the handpiece 102. An ultrasonic generator is connected to the handpiece 102, and supplies electric energy. The handpiece 102 encloses one or more ultrasonic transducers 104, which convert the supplied electric energy into ultrasonic frequency vibratory energy. The frequency range at which the system operates is typically between about 20 kHz and about 100 kHz, and the electric power supplied by the ultrasonic generator is typically between about 100 W to about 150 W. The ultrasonic transducers 104 may be made of piezoelectric material, or may be made of other materials, such as nickel, that are capable of converting electric energy into vibratory energy. The handpiece 102 typically also encloses an amplifier, for example an acoustic horn, that amplifies the mechanical vibrations generated by the ultrasonic transducers. The amplified ultrasonic energy is transmitted by horn 108 to tip assembly 110.

The ultrasonic system of the present invention is generally characterized by a resonant frequency, which is determined primarily by the assembled length of its components. The most efficient vibrations occur when the handpiece-probe assembly is vibrated at its intended resonant frequency, in which case the maximum vibratory motion occurs at the tip of the probe.

In a preferred embodiment of the invention, the system undergoes longitudinal vibratory motion, i.e. the vibrational motion is along an axis passing through the center of the ultrasonic transducer, the amplifier, and the probe member. The shape and design of the probe member significantly affect the interaction of the ultrasonic surgical system with tissue.

In one embodiment, the system includes a clamp assembly for clamping tissue between a clamping jaw and the horn. In particular, the present invention features a clamp assembly in which the ultrasonic probe member is movable, and the clamp jaw is stationary, in contrast to prior art systems which disclose stationary probe members connected to movable clamp jaws.

In one embodiment, the clamp jaw is pivotally mounted at the end of an elongated tube, and is activated by a scissors-like clamp activation mechanism.

FIGS. 2A-2D illustrate a probe-jaw assembly for an ultrasonic surgical system constructed in accordance with one embodiment of the present invention, which includes a moveable probe member having an operative surface that is substantially convex-shaped with respect to a longitudinal axis (“LA”) thereof, and a clamp jaw having an operative surface that is substantially concave-shaped with respect to a longitudinal axis (“LA”) thereof, whereby the substantially concave-shaped surface of the clamp jaw receives the substantially convex-shaped surface of the probe member when the probe member is at a closed position. In the illustrated embodiment, a pivot point is provided for the ultrasonic probe member. The pivot point is disposed at a location remote from the tip of the ultrasonic probe member.

FIG. 2A illustrates an open position of the movable probe member, in which the probe member is positioned at a location spaced apart from the clamp member. FIG. 2B illustrates a neutral position of the surgical assembly, i.e. a position in which the probe member is neither maximally spaced apart, nor closed and in engagement against the clamp member. FIG. 2C illustrates a closed position of the probe member, in which tissue can be grasped between the respective operative surfaces of the blade member and the clamp member.

FIG. 2D illustrates the stationary clamp jaw has a tissue engaging surface. The ultrasonic probe member is movably and pivotally connected to the clamp jaw. The probe member is movable between an open position spaced from the tissue engaging surface of the clamp jaw, to a clamped position in which the probe member is moved toward the tissue engaging surface so as to capture tissue therebetween. When used as a cutting instrument, the probe member may include a cutting surface that can be moved toward the tissue engaging surface of the stationary clamp jaw, so as to grasp tissue therebetween.

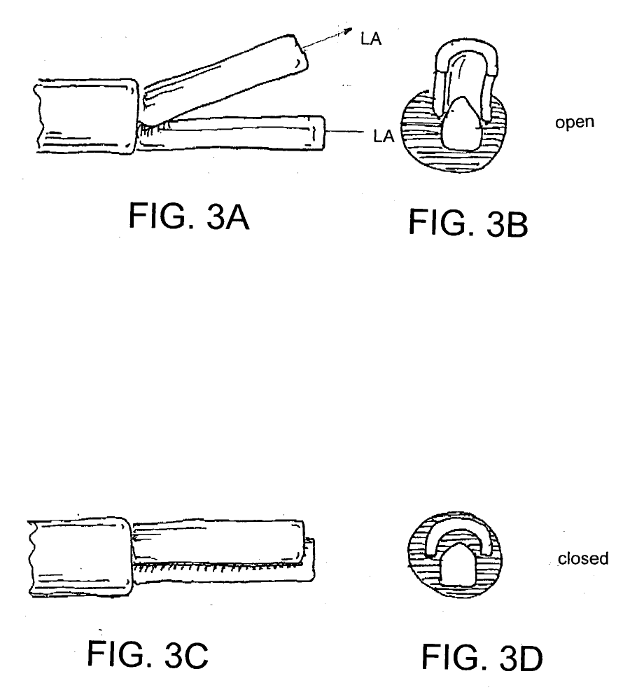

FIGS. 3A-3D illustrate another embodiment of a probe-jaw assembly for an ultrasonic surgical system, which includes a moveable probe member having an operative surface that is substantially concave-shaped with respect to a longitudinal axis (“LA”) thereof, and a clamp jaw having an operative surface that is substantially convex-shaped with respect to a longitudinal axis (“LA”) thereof, whereby the substantially concave-shaped surface of the probe member receives the substantially convex-shaped surface of the clamp jaw when the probe member is at a closed position.

FIG. 3A illustrates an open position of the movable probe member, in which the probe member is positioned at a location spaced apart from the clamp member, while FIG. 3B provides an end view thereof

FIG. 3C illustrates a closed position of the moveable probe member, in which tissue can be grasped between the respective operative surfaces of the blade member and the clamp member, while FIG. 3D provides an end view thereof.

In the illustrated embodiment, the probe member and the receiving jaw have a substantially curvilinear, non-parallel configurations. For example, a curved probe member can be optimized to produce a more uniform vibratory displacement pattern, thereby delivering a substantially uniform cutting/coagulation energy to the tissue along the entire length of the operative surface of the probe member. Also, a curvilinear probe-and-jaw assembly, having a receiving jaw that is offset and nonparallel to the ultrasonic probe member, has a greater tissue-grasping potential, as compared to linear, and/or parallel probe-and-clamp assemblies.

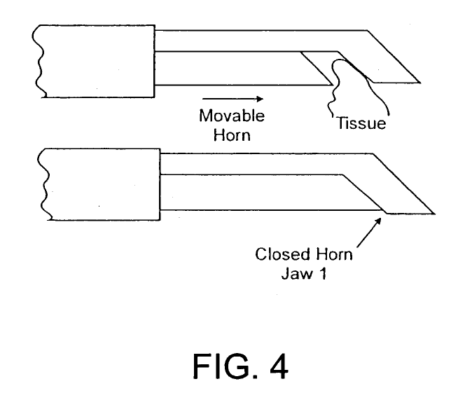

FIG. 4 illustrates a probe-jaw assembly for an ultrasonic surgical system constructed in accordance with another embodiment of the present invention, in which the ultrasonic probe member is movable in a direction parallel to the longitudinal vibrations of the probe. The probe member is connected to a fixed clamp jaw so that when the probe member is moved in the direction of longitudinal vibrations, the probe member aligns in compression against the fixed jaw. In this way, tissue disposed between the movable probe member and the fixed jaw is compressed, when the horn is moved toward the jaw.

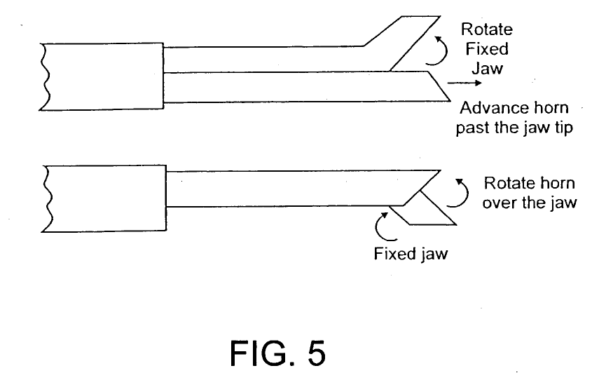

FIG. 5 illustrates a probe-jaw assembly for an ultrasonic surgical system constructed in accordance with another embodiment of the present invention, in which the ultrasonic probe member can be moved rotatably with respect to the fixed jaw. In this embodiment, the clamp jaw is stationary, but can be rotated between a plurality of different positions. After rotating the clamp jaw to a desired position, the ultrasonic probe member can be moved so as to be advanced past the tip of the clamp jaw. The probe member can then be rotated over the fixed clamp jaw.

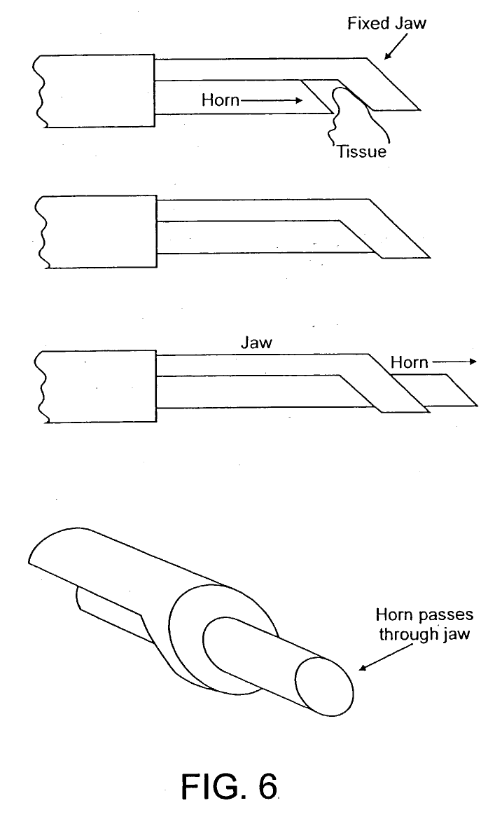

FIG. 6 illustrates a probe-jaw assembly for an ultrasonic surgical system constructed in accordance with another embodiment of the present invention, in which the ultrasonic probe member can be moved so as to pass through a matching orifice in the fixed jaw. Just as in the embodiment illustrated in FIG. 4, the ultrasonic probe member in the embodiment illustrated in FIG. 6 is movable in a direction parallel to the longitudinal vibrations of the probe, so that tissue disposed between the movable probe member and the fixed jaw is compressed, when the horn is moved toward the jaw. In the embodiment illustrated in FIG. 6, a matching orifice is provided in the receiving clamp jaw, so as to allow the movable ultrasonic probe member to pass through the orifice in the jaw as the probe member is moved toward the jaw.

By providing an ultrasonically vibrating probe member that is movable with respect to a fixed clamp jaw, the present invention provides an ultrasonic soft tissue cutting and coagulating system that is more versatile than prior art systems. For example, a wider range of ultrasonic vibrational frequency can be implemented, to achieve more diverse surgical effects.

While the invention has been particularly shown and described with reference to specific preferred embodiments, it should be understood by those skilled in the art that various changes in form and detail may be made therein without departing from the spirit and scope of the invention as defined by the appended claims.

What is claimed is:

1. An ultrasonic instrument for cutting tissue, comprising:
a. an ultrasonic transducer for generating ultrasonic vibrations;
b. a probe member connected to said ultrasonic transducer for receiving ultrasonic vibrations therefrom; and
c. a stationary clamp jaw having a tissue engaging surface;
wherein said probe member is movably connected to said clamp jaw; and
wherein said probe member includes a cutting surface movable between an open position spaced from the tissue engaging surface of the clamp jaw, to a clamped position in which the cutting surface is moved toward the tissue engaging surface so as to capture tissue therebetween.

2. An ultrasonic instrument for coagulating tissue, comprising:
a. an ultrasonic transducer for generating ultrasonic vibrations;
b. a probe member connected to said ultrasonic transducer for receiving ultrasonic vibrations therefrom; and
c. a stationary clamp jaw having a tissue engaging surface;
wherein said probe member is movably connected to said clamp jaw; and
wherein said probe member is movable between an open position spaced from the tissue engaging surface of the clamp jaw, to a clamped position in which the probe member is moved toward the tissue engaging surface so as to capture tissue therebetween.
